(19) United States
(12) Patent Application Publication
(10) Pub. No.: US 2007/0265164 A1
(43) Pub. Date: Nov. 15, 2007

(54) Active Substances for Increasing Pathogenic Defense in Plants and Methods for the Detection Thereof

(75) Inventors: Klaus Bartsch, Konigstein (DE); Arno Schulz, Eppstein (DE)

Correspondence Address: STERNE, KESSLER, GOLDSTEIN & FOX P.L.L.C., 1100 NEW YORK AVENUE, N.W., WASHINGTON, DC, 20005, US

(73) Assignee: Bayer Cropscience GmbH, Frankfurt (DE)

(21) Appl. No.: 11/632,825

(22) PCT Filed: Jul. 7, 2005
(86) PCT No.: PCT/EP05/07344

§ 371 Date: Jul. 16, 2007

(30) Foreign Application Priority Data
Jul. 20, 2004 (DE) 10 2004 035 137.6

(51) Int. Cl. A01N 25/32; A01N 37/18; A01N 43/40; A01N 43/647; A01P 21/00; C12Q 1/68

(52) U.S. Cl. 504/105; 435/6; 504/103; 504/110; 504/244; 504/261; 504/317; 504/337

(57) ABSTRACT

The invention relates to a method for finding compounds which induce plant pathogen defense, the enhanced expression of individual, or a plurality of, endogenous plant genes from the group consisting of jasmonic acid biosynthesis, plant proteinase inhibitors, plant xylanase inhibitors, plant PR proteins (pathogen-related proteins) and plant chitinases being regarded as a sign that induction has taken place, and to the use of these compounds alone or in combination with known compounds which act specifically and directly against phytopathogens, it being possible to carry out the application either simultaneously or staggered.

The invention relates to a method for finding compounds which induce plant pathogen defense, the enhanced expression of individual, or a plurality of, endogenous plant genes from the group consisting of jasmonic acid biosynthesis, plant proteinase inhibitors, plant xylanase inhibitors, plant PR proteins (pathogen-related proteins) and plant chitinases being regarded as a sign that induction has taken place, and to the use of these compounds alone or in combination with known compounds which act specifically and directly against phytopathogens, it being possible to carry out the application either simultaneously or staggered.

It is known that plants respond to natural stress conditions such as, for example, cold, heat, drought, wounding, pathogen attack (viruses, bacteria, fungi, insects) and the like, but also to herbicides, with specific or unspecific defense mechanisms [Pflanzenbiochemie, pp. 393-462 , Spektrum Akademischer Verlag, Heidelberg, Berlin, Oxford, Hans W. Heldt, 1996.; Biochemistry and Molecular Biology of Plants, pp. 1102-1203, American Society of Plant Physiologists, Rockville, Md., eds. Buchanan, Gruissem, Jones, 2000]. In this context, signal substances, for example cell wall constituents which have been generated by wounding, or specific signal substances which originate from the pathogen, act as inductors of plant signal transduction chains which eventually lead to the formation of defense molecules directed against the stressor. These can take the form of, for example, (a) low-molecular-weight substances such as, for example, phytoalexins, (b) non-enzymatic proteins such as, for example, pathogen-related proteins (PR proteins), (c) enzymatic proteins such as, for example, chitinases, glucanases, or (d) specific inhibitors of essential proteins such as, for example, protease inhibitors, xylanase inhibitors, and these substances either attack the pathogen directly or interfere with its proliferation (Dangl and Jones, 2001, Nature 411: 826-833; Kessler and Baldwin, 2003, Annual Review of Plant Biology, 53: 299-328).

An additional defense mechanism is what is known as the hypersensitive reaction (HR), which is mediated via oxidative stress and which leads to the death of plant tissue around an infection focus, which prevents the spreading of plant pathogens which depend on live cells [Pennazio, 1995, New Microbiol. 18, pp. 229-240].

In the further course of an infection, signals are transmitted, by plant messenger substances, into noninfected tissue, where, again, they result in defense reactions being triggered and interfere with the formation of secondary infections (Systemic acquired resistance, SAR) [Ryals et al., 1996, The Plant Cell 8: 1809-1819].

A series of endogenous plant signal substances which are involved in stress tolerance or pathogen defense are already known. The following may be mentioned: salicylic acid, benzoic acid, jasmonic acid or ethylene [Biochemistry and Molecular Biology of Plants, pp. 850-929, American Society of Plant Physiologists, Rockville, Md., eds. Buchanan, Gruissem, Jones, 2000]. Some of these substances or their stable synthetic derivatives and derived structures are also effective when applied externally to plants or as seed coating and activate defense reactions which result in an enhanced stress or pathogen tolerance of the plant. [Sembdner, and Parthier, 1993, Ann. Rev. Plant Physiol. Plant Mol. Biol. 44: 569-589]. The salicylate-mediated defense is directed especially against phytopathogenic fungi, bacteria and viruses [Ryals et al., 1996, The Plant Cell 8: 1809-1819].

A known synthetic product with an action comparable to that of salicylic acid, and which is capable of exerting a protective effect against phytopathogenic fungi, bacteria and viruses, is benzothiadiazole (trade name Bion®) [Achuo et al., 2004, Plant Pathology 53 (1): 65-72].

Other compounds which belong to the group of the oxylipins such as, for example, jasmonic acid, and the protective mechanisms which they trigger, are especially active against harmful insects [Walling, 2000, J Plant Growth Regul. 19, 195-216].

Thus, it is known that plants have available a plurality of endogenous reaction mechanisms which are capable of bringing about an effective defense against a wide range of harmful organisms and/or natural abiotic stress. However, a prediction as to which defense reactions can be caused specifically by the application of active ingredients has not been known to date.

There is therefore a need for a method for the specific finding of molecular activators of endogeneous plant defense mechanisms against harmful organisms and/or natural abiotic stress (such as, for example, heat, cold, drought, salinity, and acid/alkali stress), whereby novel active ingredients can be found, novel characteristics of known active ingredients with a different type of action can be identified, or else known molecules or lead structures can be optimized for use as inductors of the endogenous plant defense mechanisms.

### DEFINITIONS OF TERMS USED HEREINBELOW

The term “Blast analyses” (Blast=Basic Local Alignment Search Tool) as used in the present context describes the use of suitable computer programs for the classification, and finding, of sequences which are potentially homologous (Altschul et al., J. Mol. Biol. 1990, 215: 403-410), a comparison (alignment) between a query sequence and all the sequences of one or more databases taking place, with a desired agreement being set in the form of “significance criteria” (scoring function) (R. Rauhut, Bioinformatik, pp. 38-107, Verlag Wiley-VCH Verlag GmbH, Weinheim, 2001).

The term “cDNA” (complementary DNA) as used in the present context describes a DNA single strand which is complementary to an RNA and which is synthesized in vitro by an enzymatic reverse transcription. The cDNA can either correspond to the full length of the RNA or else only constitute a part-sequence of the RNA which acts as template.

The term “cluster analysis” as used in the present context means that the individual data which have been determined are clustered by means of a computer program which has been developed for this purpose, with groups of genes which code for proteins with a similar function, or else genes with a similar expression pattern, are shown as clusters. A hierarchic minimization of the complex data pattern, which can be shown in the form of a dendrogram, is thereby achieved. Cluster analysis makes possible the classifying evaluation of the data sets obtained, which is much more than a mere accumulation of data which are not interrelated.

The terms “DNA chip”, “DNA array” and “DNA microarray”, which are used synonymously in the present context, is understood as meaning a support whose base comprises for example glass or nylon, whose base has DNA fragments attached to it, where it is possible for the DNA to be applied for example by (a) a photolithographic process (DNA is synthesized directly on the support of the array), (b) a microspotting method (externally synthesized oligonucleotides or PCR products are applied to the support, where they are bonded covalently) or (c) by a microspraying method (externally synthesized oligonucleotides or PCR products are sprayed onto the support using an ink-jet printer, without touching) (R. Rauhut, Bioinformatik, pp. 197-199, Verlag Wiley-VCH Verlag GmbH, Weinheim, 2001). A DNA chip which represents the genomic sequences of an organism is referred to as a “genomic DNA chip”. The evaluation of the data obtained with the aid of these “DNA chips” is referred to as “DNA chip analysis”.

The term “DNA chip hybridization” as used in the present context means the pairing of two single-stranded complementary nucleic acid molecules, one of the base-pairing molecule partners being localized as DNA (deoxyribonucleic acid) on the DNA chip, preferably in covalently bonded form, while the other, in the form of the RNA (ribonucleic acid) or the corresponding cDNA (complementary DNA) is in solution. The hybridization of the bonded and unbonded nucleic acids takes place on the DNA chip in an aqueous buffer solution, if appropriate under additionally denaturing conditions, such as, for example, in the presence of dimethyl sulfoxide, at a temperature of 30-60° C., preferably 40-50° C., especially preferably at 45° C. for 10-20 hours, preferably for 14-18 hours, especially preferably for 16 hours, with constant shaking. The hybridization conditions can be established in such a way that they are constant, for example in a hybridization oven. In such a hybridization oven, shaking at 60 rpm (rounds per minute, or revolutions per minute) is routinely performed.

The nucleic acid sequence in the present context, for which the term “EST sequence” (expressed sequence tag) is used, means a short sequence of 200-500 bases or base pairs.

As used in the present context, the terms “expression pattern”, “induction pattern” and “expression profile”, which are used synonymously, describe the temporarily differentiated and/or tissue-specific expression of the plant mRNA, the pattern being obtained directly, with the aid of DNA chip technology, by the generated intensity of the hybridization signal of the RNA obtained from the plant, or its corresponding cDNA. The “expression values” measured are the result of direct computation with the corresponding signals which are obtained by using a synonymous chip and hybridization of an untreated control plant.

As used in the present context, the term “expression state”, which is obtained by the gene expression profiling performed, describes all of the recorded transcription activity of cellular genes which is measured with the aid of a DNA chip.

As used in the present context, the term “total RNA” describes the representation, which is possible as the result of the extraction method employed, of different endogenous plant RNA groups which may be present in a plant cell, such as, for example, cytoplasmic rRNA (ribosomal RNA), cytoplasmic tRNA (transfer RNA), cytoplasmic mRNA (messenger RNA), and their respective nuclear precursors, ctRNA (chloroplastidic RNA) and mtRNA (mitochondrial RNA), but it also comprises RNA molecules which may originate from exogenous organisms, such as, for example, from viruses or parasitic bacteria and fungi.

As used in the present context, the term “useful plants” refers to crop plants which are used as plants for obtaining foods, feedstuffs or for industrial purposes.

As used in the present context, the term “safener” denotes a chemical compound which is not of endogenous plant origin and which cancels or lessens the phytotoxic effects of a pesticide on crop plants, without substantially reducing the pesticidal activity against harmful organisms such as, for example, weeds, bacteria, viruses and fungi.

The present invention relates to a method for finding a compound which induces plant pathogen defense, the enhanced transcription or expression of individual, or a plurality of, endogenous plant genes from the group consisting of jasmonic acid biosynthesis, plant proteinase inhibitors, plant xylanase inhibitors, plant PR proteins (pathogen-related proteins) and plant chitinases being regarded as a sign that induction has taken place.

The present invention especially relates to a method for finding compounds which induce the transcription of the genes coding for endogenous plant pathogen-defense enzymes, wherein:
a) test plants are brought into contact with a suitable amount of the test substance(s),
b) control plants are, under otherwise identical conditions as test plants of a), not brought into contact with the test substance(s),
c) RNA is extracted from the test plants and the control plants,
d) the RNA is either radiolabeled directly or else not radiolabeled, or else the RNA is radiolabeled or nonradiolabeled while simultaneously being transcribed enzymatically into the corresponding cDNA, or else the resulting, unlabeled cDNA is transcribed enzymatically into a corresponding radiolabeled or non-radiolabeled cRNA,
e) a DNA array comprising plant DNA sequences is hybridized with the substances obtained in step d),
f) expression profiles of the genes for the expression of jasmonic acid biosynthesis, plant proteinase inhibitors, plant xylanase inhibitors, plant PR proteins (pathogen-related proteins) and/or plant chitanases are generated in a comparative manner for the plants tested as described in a) and b),
g) the expression differentials measured in f) are quantified, and
h) the expression profiles assigned as described in g) are subjected to a final systematic grouping by means of cluster analysis.

In the case of the abovementioned step d), the enzymatic transcription of the resulting cDNA into a cRNA must be considered as a preferred process step since this allows the hybridization probe to be amplified yet again. Likewise preferred is labeling by means of nonradioactive nucleotides, especially preferably labeling by means of biotinylated UTP and/or CTP, where, once the hybridization reaction has been carried out, detection takes place by the binding of streptavidin-phycoerythrin, as fluorophor, to the biotinylated cRNA. After the hybridization, the specific phycoerythrin fluorescence, which acts as the basis for the quantification of the expression differentials measured, is detected with the aid of a laser scanner.

Preferred subject matter of the present invention is a method in which the abovementioned process steps a)-h) are adhered to, with
(i) the expression profile of a gene of the lipase-like protein, of 12-oxophytodienoate reductase (EC 1.3.1.42), of allene-oxide cyclase, of 12-oxophytodienoic acid reductase, and/or
(ii) the expression profile of a gene of a plant proteinase inhibitor which has significant homologies with the proteinase inhibitor with the PIR protein database entry S71555, and/or
(iii) the expression profile of a gene of a plant xylanase inhibitor protein, and/or
(iv) the expression profile of a gene of the pathogen-induced plant peroxidase (EC 1.11.1.7), a protein with significant homologies to the barley pathogen-related (PR) protein with the number T06168 in the PIR protein database, and/or
(v) the expression profile of a gene of the plant chitinase
   being enhanced in comparison with an untreated control plant, for example by the factor 2 or more, preferably by the factor 2-100, preferably 2-20, especially preferably 2-10, very especially preferably 2-5, and with it being possible for the enhancement of the modified expression profiles of the individual genes independently of one another to be in the different abovementioned ranges.

The present invention furthermore relates to the use of certain DNA microarrays which, based on genetic information from plants, preferably genetic information from useful plants, especially preferably from useful plants such as, for example, barley, maize, wheat, rice, oats, oilseed rape, sugar beet, are utilized for finding modified gene expression patterns. In this context, the relative modifications of the gene patterns for genes of jasmonic acid biosynthesis, plant proteinase inhibitors, plant xylanase inhibitors, plant PR proteins (pathogen-related proteins) and/or plant chitinases are observed in plants which have been treated with the compounds to be tested in comparison with untreated control plants.

The present invention furthermore also relates to the use of the compounds which have been identified in the abovementioned method as being positive, i.e. enhancing the expression with regard to their inductive effect on genes of jasmonic acid biosynthesis, plant proteinase inhibitors, plant PR proteins (pathogen-related proteins) and/or plant chitinases as active ingredients for enhancing stress tolerance and/or pathogen defense in useful plants.

The present invention therefore also relates to the use of compounds which, in plants, contribute directly or indirectly, such as, for example, by a signal transduction chain, to an enhanced defense against phytopathogenic organisms such as, for example, insects, fungi, bacteria or viruses, with at least one gene, preferably more than one gene for proteins from the group of the proteins of jasmonic acid biosynthesis, plant proteinase inhibitors, plant xylanase inhibitors, plant PR proteins, (pathogen-related proteins) and/or plant chitinases, having an enhanced expression profile.

Compounds whose use as what are known as safeners is already known in crop protection such as, for example, mefenpyr-dimethyl, mefenpyr-diethyl, mefenpyr analogs, isoxadifen-ethyl, cloquintocet-mexyl, cloquintocet derivatives and pyridinecarboxamide, are preferred in this context.

By applying the abovementioned compounds, it is possible to protect crop plants efficiently against phytopathogenic organisms (insects, fungi, bacteria, viruses), which also has an effect on, for example, higher yields. An advantage over active ingredients which are directed directly against these organisms is that beneficial creatures are left unharmed since they do not trigger the defense reactions in question.

The present invention thus also relates to a method for protecting useful plants in crops of useful plants against phytopathogenic organisms, especially against insects, fungi, bacteria and viruses, by applying the compounds which have been identified with the aid of the DNA array, taking into consideration the expression profiles of the genes of jasmonic acid biosynthesis, plant proteinase inhibitors, plant xylanase inhibitors, plant PR proteins (pathogen-related proteins) and/or plant chitinases. Very especially preferred is the protection against phytopathogenic organisms, and in particular against insects, very particularly against sucking insects.

The abovementioned mechanisms which have been triggered by the compounds identified with the aid of the DNA arrays, such as also for example compounds which are already known as safeners, also lead to synergistic effects between these compounds and further compounds which act specifically and directly against phytopathogens, such as, for example, insecticides and fungicides, especially when applied simultaneously or staggered with fungicides and insecticides, especially insecticides, such as, for example, compounds from the group of the ketoenols [for example in EP 528156; EP 596298], or agonists or antagonists of the nicotinergic acetylcholine receptor. Some of the last-mentioned compounds are grouped under the term nitromethylenes or nitroimines and related compounds (for example in EP 464830).

The present invention therefore also relates to the use of the compounds identified by screening by means of a DNA array taking into consideration the expression profiles of the genes of jasmonic acid biosynthesis, plant proteinase inhibitors, plant xylanase inhibitors, plant PR proteins (pathogen-related proteins) and/or plant chitinases in combination with priorart compounds which act specifically and directly against phytopathogens, such as, for example, the combination of mefenpyr derivatives (such as, for example, the compound listed as No. 506 in “The Pesticide Manual, 13^{th} Edition, 2003”) with insecticides which act directly on the harmful organism, especially preferably with an insecticide from the group of the ketoenols or the group of the nitromethylenes/nitroimines, or the use of isoxadifen derivatives (such as, for example, the compound listed as No. 478 in “The Pesticide Manual, 13^{th} Edition, 2003”) with insecticides which act directly on the harmful organism, it being possible for the application to take place either simultaneously or staggered. When the application does not take place simultaneously, the insecticide which acts directly on the harmful organism can be applied either before or after the application of the compound identified by means of the DNA array described herein has taken place, with subsequent application of the insecticide which acts directly on the harmful organism being preferred.

The examples which follow describe the invention in detail.

### EXAMPLE 1

Detection of the Effects of Safeners on Defense Mechanisms in Plants by Gene Expression Profiling (GEP):

Barley plants (cv. Baronesse) were grown in compost-filled pots (diameter: 10 cm) for 10 days in a controlled-environment cabinet under defined light, humidity and temperature conditions (white light, 70% atmospheric humidity, 24° C.). At the time of spray application, the seedlings were approximately 15 cm in size. The test substances selected in this example were molecules which differ markedly in their structure and which have a well documented pronounced safener effect, and other molecules which are known from the literature to have an effect on plant stress and pathogen tolerance (Table 1). The substances were prepared as stock solutions in DMSO (dimethyl sulfoxide) at c=10 mg/ml). This was used for preparing dilutions in water supplemented with 0.2% Agrotin (comprises polyvinyl alcohol, silicones, polysaccharides and pH regulators, manufacturer: Bayer CropScience AG, Monheim, Germany), as wetter, to give the application rates shown in the table. The amount of liquid for the spray application was 800 μl per pot, corresponding to 800 l/ha. For each 800 μl of spray mixture, 16 μl of EC premix: diacetone alcohol=1:6 were additionally added as formulation auxiliaries. The substances listed in Table 1 were applied to the leaves by means of a pneumatic spray pistol. Each substance was sprayed as 2 dosages, and all experiments were carried out as replicates (2 pots per substance). Sprayings with blank formulation without active ingredient were carried out as controls. After 6 hours, the leaves were harvested, frozen in liquid nitrogen and stored at −80° C. until further use. The labeled RNA probes for the DNA chip hybridization were prepared as described in the protocols (Expression Analysis, Technical Manual) from Affymetrix (Affymetrix Inc., 3380 Central Expressway, Santa Clara, Calif., USA). First, total RNA was isolated from in each case 500 mg of the harvested leaves. In each case 10 μg of total RNA was used for the first-strand and second-strand cDNA synthesis. The cDNA was amplified with T7 polymerase and simultaneously labeled with biotin-UTP. In each case, 20 μg of this biotinylated cDNA was employed for the hybridization of the barley genome array (Gene Chip Barleyl, order No.: 511012) from Affymetrix. This DNA microarray comprises DNA sequences of 22 840 genes which are composed of a total of 400 000 EST sequences. Thereafter, the DNA microarrays were washed in the Affymetrix Fluidics Station, stained with streptavidin/phycoerythrin (Molecular Probes, P/N S-866) and scanned with the matching Agilent Laser Scanner (Agilent Gene Array Scanner). The fluorescent data obtained were analyzed with the software Microarray Suite 5 from Affymetrix. After the quality had been checked, all DNA chip analyses were stored in a database. To determine the relative expression values for genes (induction factors, repression factors), test and control chips were compared with each other, and the scoring function set by the Affymetrix software was used as the basis. The 2 biological replications of one experiment were compared in each case with 2 control chips (blank formulation), and the 4 expression values obtained for each gene were used for calculating the median. These medians are shown in the results tables as induction factors. Similarity comparisons of expression profiles from different experiments and cluster analyses were carried out using the software GeneMaths 1.5 from Applied Maths (Applied Maths, Keistraat 120, 9830 Sint-Martens-Latem, Belgium).

Gene groups from specific metabolic pathways and signal transduction chains were put together by key word search in the annotations, of the genes, which were also provided by Affymetrix, and by Blast analyses (homology comparisons) of the DNA target sequences, i.e. of the positive sequence (identified on the basis of a modified expression profile) identified on the DNA chip, using gene sequences from other organisms, preferably plant organisms, with characterized functions.

TABLE 1

| | Application rate | |
|---|---|---|
| Test substance (No.): | [g a.i./ha]: | Known property: |
| Mefenpyr-dimethyl (1) | 150 | safener |
| Mefenpyr analogue (2) | 150 | safener |
| Isoxadifen-ethyl (3) | 150 | safener |
| Cloquintocet-mexyl (4) | 150 | safener |
| Cloquintocet derivative (5) | 150 | safener |
| Pyridinecarboxamide (6) | 150 | safener |
| Salicyl hydroxamate (7) | 500 | resistance inductor |
| Bion ® (=Acibenzolar-S- | 150 | resistance inductor |
| methyl) (8) | | |
| Dichloroisonicotinic acid (9) | 150 | resistance inductor |
| Dichlorosalicylic acid (10) | 150 | resistance inductor |

Structural Formulae of the Test Substances Used, as shown in Table 1 Above:

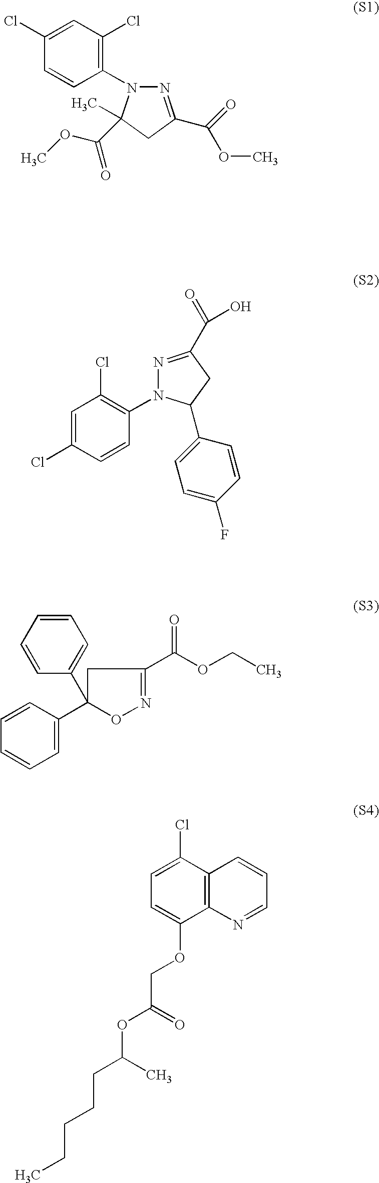

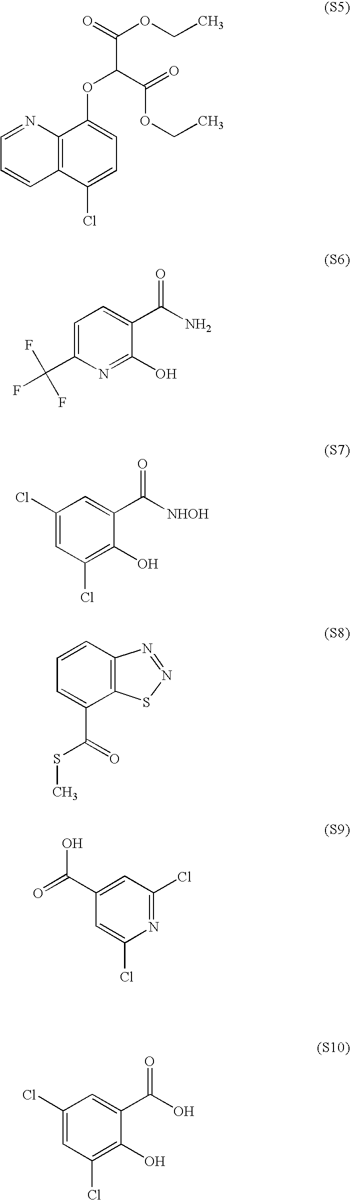

Screening gene groups from signal transduction chains and metabolic pathways which play a role in stress tolerance and pathogen defense revealed, inter alia, a high level of induction of genes for protease and xylanase inhibitors and of jasmonic acid biosynthesis genes (Table 2a), and genes for PR proteins and chitinases, (Table 2b), a group of compounds already known for their safener effect (S1-S6).

TABLE 2a

| “Probe Set” Number | S1 | S2 | S3 | S4 | S5 | S6 | S7 | S8 | S9 | S10 |
|---|---|---|---|---|---|---|---|---|---|---|
| 1.1 Contig2631_at | 1.00 | 1.00 | 97.68 | 1.00 | 1.00 | 1.00 | 1.00 | 1.00 | 1.00 | 1.00 |
| 1.2 HI02E21u_s_at | 1.00 | 1.00 | 40.79 | 1.00 | 1.00 | 1.00 | 1.00 | 1.00 | 3.03 | 4.66 |
| 1.3 Contig3097_at | 1.00 | 1.00 | 8.51 | 1.00 | 1.00 | 1.00 | 1.00 | 2.11 | 1.00 | 1.00 |
| 1.4 Contig5146_at | 7.73 | 4.47 | 4.50 | 4.72 | 5.35 | 6.77 | 1.00 | 1.00 | 1.00 | 1.00 |
| 1.5 Contig 4986_at | 1.00 | 1.00 | 3.76 | 1.00 | 1.00 | 1.00 | 1.00 | 1.00 | 1.00 | 1.00 |
| 1.6 HV_Ceb0020 | 1.00 | 1.00 | 3.68 | 1.00 | 1.00 | 1.00 | 1.00 | 1.00 | 1.00 | 1.00 |
| D05r2_s_at | | | | | | | | | | |
| 1.7 Contig6194_a_at | 12.91 | 4.14 | 3.25 | 2.17 | 2.81 | 3.16 | 1.00 | 1.00 | 1.00 | 1.00 |
| 1.8 Contig9556_at | 1.00 | 1.00 | 3.03 | 1.00 | 1.00 | 1.00 | 1.00 | 1.00 | 1.00 | 1.00 |
| 1.9 Contig15_a_at | 0.35 | 1.00 | 2.97 | 1.00 | 0.46 | 1.00 | 1.00 | 1.00 | 1.00 | 1.00 |
| 1.10 Contig1736_at | 1.00 | 1.00 | 2.91 | 1.00 | 1.00 | 1.00 | 1.00 | 1.00 | 1.00 | 1.00 |
| 1.11 Contig12574_at | 0.50 | 1.00 | 2.81 | 1.00 | 1.00 | 1.00 | 1.00 | 2.11 | 1.00 | 1.00 |
| 1.12 Contig6611_at | 1.00 | 1.00 | 2.41 | 1.00 | 1.00 | 1.00 | 2.31 | 3.61 | 2.00 | 2.38 |
| 1.13 Contig10030_at | 1.00 | 1.00 | 2.25 | 1.00 | 1.00 | 1.00 | 1.00 | 1.00 | 1.00 | 1.00 |
| 1.14 Contig2305_at | 1.00 | 1.00 | 2.14 | 1.00 | 1.00 | 1.00 | 41.07 | 81.57 | 12.55 | 18.13 |
| 1.15 Contig1737_at | 1.00 | 1.00 | 1.00 | 1.00 | 1.00 | 1.00 | 1.00 | 1.00 | 1.00 | 1.00 |
| 1.16 Contig3096_s_at | 1.00 | 1.00 | 1.00 | 1.00 | 1.00 | 1.00 | 2.06 | 2.68 | 1.00 | 1.00 |
| 1.17 Contig13413_at | 1.00 | 1.00 | 1.00 | 1.00 | 1.00 | 1.00 | 1.00 | 2.17 | 1.00 | 1.00 |
| 1.18 Contig2326_s_at | 2.31 | 1.00 | 1.00 | 2.45 | 1.00 | 1.00 | 1.00 | 1.00 | 1.00 | 1.00 |
| 1.19 Contig2330_x_at | 4.03 | 2.39 | 1.00 | 2.83 | 1.00 | 2.55 | 1.00 | 1.00 | 1.00 | 1.00 |
| 1.20 Contig2337_at | 4.89 | 1.00 | 1.00 | 1.00 | 1.00 | 1.00 | 1.00 | 1.00 | 1.00 | 1.00 |
| 2.1 Contig2088_s_at | 1.00 | 1.00 | 4.26 | 1.00 | 1.00 | 1.00 | 4.79 | 7.36 | 2.36 | 2.41 |
| 2.2 Contig34_s_at | 1.00 | 1.00 | 1.00 | 1.00 | 1.00 | 1.00 | 4.50 | 3.68 | 1.00 | 2.08 |
| 2.3 Contig88_x_at | 1.00 | 1.00 | 4.53 | 1.00 | 1.00 | 1.00 | 21.86 | 22.94 | 4.59 | 4.41 |
| 2.4 HD07M22r_s_at | 6.32 | 1.00 | 2.71 | 1.00 | 7.84 | 5.10 | 1.00 | 1.00 | 1.00 | 1.00 |
| 3.1 Contig8905_at | 3.10 | 2.53 | 2.99 | 2.73 | 3.01 | 2.45 | 1.00 | 1.00 | 2.53 | 1.00 |

Expression values (x times above the value of the untreated control) measured for:

(a) genes of plant jasmonic acid biosynthesis (1.1-1.20)

(b) genes which code for plant proteinase inhibitors (2.1-2.4)

(c) gene which codes for a plant xylanase inhibitor (3.1)

The “probe set” number corresponds to the respective DNA chip position of the Affymetrix chip.

Using a Blast analysis, the best-possible corresponding known sequence from other annotated sequence databases can be assigned to the “probe set” number. These data which are shown in the Blast analysis are shown hereinbelow.

| | |
|---|---|
| | corresponding sequence with annotated function |
| “Probe Set Number” | from publicly accessible DNA or protein database |
| 1.1 | Lipase-like Protein (Oryza sativa; japonica |
| | cultivar group) |
| 1.2 | Lipoxygenase (EC 1.13.11.12) barley gbAAB70865 |
| 1.3 | Allene oxide synthase (Horedeum vulgare subsp. |
| | vulgare) |
| 1.4 | Probable 12-oxophytodienoate reductase |
| | (EC 1.3.1.42) |
| 1.5 | Allene oxide cyclase (Oryza sativa; japonica |
| | cultivar group) |
| 1.6 | Allene oxide cyclase (Oryza sativa; japonica |
| | cultivar group) |
| 1.7 | 12-oxophytodienoic acid reductase (Oryza sativa) |
| 1.8 | 12-oxophytodienoate reductase 3 (Lycopersicon |
| | esculentum) |
| 1.9 | GDSL-motif lipase/hydrolase-like protein, |
| | At5g55050.1 |
| 1.10 | Lipoxygenase 1 pir T05941 (EC 1.13.11.12) |
| | barley |
| 1.11 | Putative lipoxygenase (Oryza sativa, japonica |
| | cultivar group) |
| 1.12 | Similar to lipases (Arabidopsis thaliana; |
| | gb AAM20450.1) |
| 1.13 | Putative lipase homolog (Oryza sativa; |
| | japonica cultivar group) |
| 1.14 | Methyljasmonate inducible lipoxygenase |
| | gbAAC12951.1 |
| 1.15 | Probable lipoxygenase gbAAB60715.1 |
| 1.16 | Allene oxide synthase (Hordeum vulgare subsp. |
| | vulgare) |
| 1.17 | Similar to lipases (Arabidopsis thaliana; |
| | gb AAM20450.1) |

| | |
|---|---|
| | corresponding sequence with annotated function |
| “Probe Set Number” | from publicly accessible DNA or protein database |
| 1.18 | 12-oxophytodienoic acid reductase (Oryza sativa) |
| 1.19 | 12-oxophytodienoic acid reductase (Oryza sativa) |
| 1.20 | 12-oxophytodienoate reductase (OPR1) |
| | At1g76680.1 |

| | corresponding sequence with annotated function |
|---|---|
| “Probe Set Number” | from publicly accessible DNA or protein database |
| 2.1 | Bowman-Birk type trypsin inhibitor |
| 2.2 | Putative proteinase inhibitor (Hordeum vulgare |
| | subsp. vulgare) |
| 2.3 | Putative proteinase inhibitor (Hordeum vulgare |
| | subsp. vulgare) |
| 2.4 | Proteinase-Inhibitor (pir S71555, barley) |
| 3.1 | Xytanase inhibitor protein (Triticum aestivum) |

TABLE 2b

| “Probe Set”- Number | S1 | S2 | S3 | S4 | S5 | S6 | S7 | S8 | S9 | S10 |
|---|---|---|---|---|---|---|---|---|---|---|
| 4.1 Contig2118_at | 8.69 | 1.00 | 4.86 | 1.00 | 2.33 | 2.20 | 1.00 | 1.00 | 1.00 | 1.00 |
| 4.2 Contig5369_at | 2.35 | 1.00 | 4.76 | 1.00 | 1.00 | 1.00 | 1.00 | 1.00 | 1.00 | 1.00 |
| 4.3 Contig5607_s_at | 18.64 | 15.56 | 13.36 | 18.38 | 8.75 | 9.99 | 1.00 | 1.00 | 1.00 | 1.00 |
| 5.1 Contig2990_at | 4.17 | 1.00 | 2.77 | 1.00 | 1.00 | 1.00 | 1.00 | 1.00 | 2.39 | 1.00 |
| 5.2 Contig2992_s_at | 6.50 | 1.00 | 2.19 | 1.00 | 3.12 | 2.27 | 1.00 | 1.00 | 2.14 | 1.00 |
| 5.3 Contig5023_at | 9.51 | 3.18 | 1.00 | 1.00 | 2.73 | 1.00 | 1.00 | 1.00 | 4.56 | 1.00 |

Expression values (x times above the value of the untreated control) measured for:

(a) genes which code for plant PR proteins (4.1-4.3)

(b) genes which code for plant chitinases (5.1-5.3)

The “probe set” number corresponds to the respective DNA chip position of the Affymetrix chip.

Using a Blast analysis, the best-possible corresponding known sequence from other annotated sequence databases can be assigned to the “probe set” number. These data which are shown in the Blast analysis are shown hereinbelow.

“Probe Set” Number Corresponding Sequence with Annotated Function from Publicly Accessible DNA or Protein Database

| | |
|---|---|
| | corresponding sequence with annotated function |
| “Probe Set Number” | from publicly accessible DNA or protein database |
| 4.1 | Peroxidase (EC 1.11.1.7), pathogen-induced (barley) |
| 4.2 | Pathogen-related protein; (Oryza sativa; |
| | gbAALY4406.1) |
| 4.3 | Pathogen-related protein (barely; PirT06168) |
| 5.1 | Chitinase (EC 3.2.1.14) (Barley; embCAA55344.1) |
| 5.2 | Chitinase (EC 3.2.1.14) (Barley; embCAA55344.1) |
| 5.3 | Class III chitinase (Oryza sativa subsp. japonica; |
| | gbAAM08773.1) |

The induction patterns which are derived from these tables and which are shown directly by the expression values obtained show characteristic differences between safeners and resistance inductors, with the effect on jasmonic acid biosynthesis being most pronounced in the case of isoxadifen. The expression patterns found permit substances with a similar signature to be found, and suggest that these substances have a similar type of effect in the activation of plant pathogen defense.

### EXAMPLE 2

Repellent Effect of Safener-Treated Plants on Phytopathogenic Insects:

Barley plants (cv. Baronesse) were grown as described in Example 1 and, after 10 days, treated by spray application with 150 g a.i./ha of isoxadifen-ethyl (S 3) or blank formulation. The experiments were carried out in replications of in each case 10 pots.

6 hours or 24 hours after the substance had been applied, all pots were infested uniformly with a population of the phytopathogenic aphid Rhopalosiphum padi. The experiments were evaluated after 7 days and 14 days by counting the animals on the leaves.

After 7 days, the aphid populations on the safener-treated plants averaged 50% less, and after 14 days 70% less, than the controls.

A direct toxicity of isoxadifen-ethyl (S3) against aphids was not detected in the plant-free Sachez test.

### EXAMPLE 3

Detection of Elevated Oxophytodienoate (OPDA) and Jasmonate (JA) Concentrations in Isoxadifen-Treated Plants

Barley plants (cv. Baronesse) were grown under the conditions described in Example 1 and treated by spray application with safener isoxadifen-ethyl (S 3). The application rates were chosen as follows:

(1) 30 [g a.i./ha]; (2) 150 [g a.i./ha]; (3) blank formulation (no active ingredient)

The plant leaves were harvested at different points in time after the treatment (h=hours), viz. after 1 h, 2 h, 4 h, 6 h, 12 h, 24 h and 48 h. All samples were designed in each case as replications of 3 pots.

The octadecanoates and jasmonates were processed from the leaves following a procedure described in the literature (Müller A, Düchting P and Weiler E W (2002)), A multiplex GC-MS/MS technique for the sensitive and quantitative single-run analysis of acidic phytohormones and related compounds, and its application to Arabidopsis thaliana. Planta, 216, 44-56).

300 mg of plant material were extracted for each measuring point.

50 pmol [¹³C]2-JA and 100 pmol [17,17,17,18,18-²H]-cis-OPDA were applied as internal standards. The extracts were purified via aminopropyl anion-exchange chromatography in miniature solid-phase exchanger columns which had been prepared in-house.

The results are shown in Tables 3a to 3d, the values shown being in each case means of the 3 applications.

TABLE 3a
Oxophytodienoate (OPDA) concentration measured
in pmol/g leaf tissue

| | | Isoxadifen-ethyl (S3) |
|---|---|---|
| Time/hours | Control | 30 g a.i/ha |
| 0 | 2500 | 2500 |
| 1 | 1500 | 6500 |
| 2 | 1000 | 3750 |
| 4 | 2500 | 7000 |
| 6 | 2500 | 9000 |

TABLE 3b
Oxophytodienoate (OPDA) concentration measured
in pmol/g leaf tissue

| | | Isoxadifen-ethyl (S3) |
|---|---|---|
| Time/hours | Control | 150 g a.i./ha |
| 0 | 2600 | 2600 |
| 1 | 1600 | 2800 |
| 2 | 1200 | 1800 |
| 4 | 2600 | 2200 |
| 6 | 2700 | 5100 |
| 12 | 3600 | 2400 |
| 24 | 2800 | 5100 |
| 48 | 1800 | 1900 |

TABLE 3c
Jasmonate (JA) concentration measured in pmol/g leaf tissue

| | | Isoxadifen-ethyl (S3) |
|---|---|---|
| Time/hours | Control | 30 g a.i/ha |
| 0 | 140 | 140 |
| 1 | 180 | 600 |
| 2 | 70 | 320 |
| 4 | 190 | 790 |
| 6 | 130 | 620 |

TABLE 3d
Jasmonate (JA) concentration measured in pmol/g leaf tissue

| | | Isoxadifen-ethyl (S3) |
|---|---|---|
| Time/hours | Control | 150 g a.i./ha |
| 0 | 140 | 140 |
| 1 | 175 | 290 |
| 2 | 70 | 280 |
| 4 | 190 | 270 |
| 6 | 135 | 275 |
| 12 | 220 | 155 |
| 24 | 145 | 265 |
| 48 | 135 | 320 |

The oxophytodienoate (OPDA) concentrations were in the range of 1800-9000 pmol/g leaf tissue, in the case of jasmonate (JA) in the range of 70-800 pmol/g leaf tissue. After treatment with isoxadifen-ethyl (S 3), a pronounced increase in the concentrations of both substances of up to approximately 5 times the values in the blank formulation samples without active ingredient were measured.

We claim:

1. A method for finding compounds which induce plant pathogen defense, the enhanced expression of individual, or a plurality of, endogenous plant genes from the group consisting of jasmonic acid biosynthesis, plant proteinase inhibitors, plant xylanase inhibitors, plant PR proteins (pathogen-related proteins) and plant chitinases being regarded as a sign that induction has taken place.

2. The method as claimed in claim 1, wherein
a) test plants are brought into contact with a suitable amount of the test substance(s),
b) control plants are, under otherwise identical conditions as test plants of a), not brought into contact with the test substance(s),
c) RNA is extracted from the test plants and the control plants,
d) the RNA is either radiolabeled directly or else not radiolabeled, or else the RNA is radiolabeled or nonradiolabeled while simultaneously being transcribed enzymatically into the corresponding cDNA, or else the previously obtained, unlabeled cDNA is transcribed enzymatically into a corresponding radiolabeled or non-radiolabeled cRNA,
e) a DNA array comprising plant DNA sequences is hybridized with the substances obtained in step d),
f) expression profiles of the genes for the expression of jasmonic acid biosynthesis, plant xylanase inhibitors, plant proteinase inhibitors, plant PR proteins (pathogen-related proteins) and/or plant chitanases are generated in a comparative manner for the plants tested as described in a) and b),
g) the expression differentials measured in f) are quantified, and
h) the expression profiles assigned as described in g) are subjected to a final systematic grouping by means of cluster analysis.

3. The method as claimed in claim 2, wherein:
(i) the expression profile of a gene of the lipase-like protein, of 12-oxophytodienoate reductase (EC 1.3.1.42), of allene-oxide cyclase, of 12-oxophytodienoic acid reductase, and/or
(ii) the expression profile of a gene of a plant proteinase inhibitor which has significant homologies with the proteinase inhibitor with the PIR protein database entry S71555, and/or
(iii) the expression profile of a gene of a plant xylanase inhibitor protein, and/or
(iv) the expression profile of a gene of the pathogen-induced plant peroxidase (EC 1.11.1.7), a protein with significant homologies to the barley pathogen-related (PR) protein with the number T06168 in the PIR protein database, and/or
(v) the expression profile of a gene of the plant chitinase is enhanced in comparison with an untreated control plant by the factor 2 or more.

4. The method as claimed in claim 3, wherein the expression profiles of one or more of the genes stated under (i)-(v) are enhanced by the factor 2-20.

5. The use of compounds which, in plants, contribute directly or indirectly, to an enhanced defense against phytopathogenic organisms, with at least one gene, preferably more than one gene coding for proteins from the group of the proteins of jasmonic acid biosynthesis, plant proteinase inhibitors, plant xylanase inhibitors, plant PR proteins, (pathogen-related proteins) and/or plant chitinases, having an enhanced expression profile.

6. The use of compounds as claimed in claim 5 whose use as what are known as safeners is already known in crop protection.

7. The use as claimed in claim 5 or 6, wherein the compounds have been identified as claimed in any of claims 2 to 4.

8. The use of compounds as claimed in claim 6, which are the compounds mefenpyr-dimethyl, mefenpyr-diethyl, mefenpyr analogs, isoxadifen-ethyl, cloquintocet-mexyl, cloquintocet derivatives and pyridine carboxamide.

9. The use as claimed in any of claims 5 to 8, wherein the compounds are employed in crops of useful plants in combination with prior-art compounds which act specifically and directly against phytopathogens for enhancing the defense against phytopathogenic organisms, it being possible for the application to take place simultaneously or staggered.

10. The use as claimed in claim 9, wherein the mefenpyr derivatives or isoxadifen derivatives are combined with insecticides which act directly on the harmful organism.

11. The use as claimed in claim 10, where, in the case of mefenpyr derivatives, the insecticide which acts directly on the harmful organism corresponds to a ketoenol or a nitromethylene/nitroimine.

12. A method for protecting useful plants in crops of useful plants against phytopathogenic organisms, by applying compounds which are employed in plants, directly or indirectly, for enhancing the defense against phytopathogenic organisms, with at least one, preferably more than one gene coding for proteins from the group of the proteins of jasmonic acid biosynthesis, plant xylanase inhibitors, plant proteinase inhibitors, plant PR proteins (pathogen-related proteins) and/or plant chitinases, having an enhanced expression profile.
